(19) United States
(12) Patent Application Publication
(10) Pub. No.: US 2004/0127802 A1
(43) Pub. Date: Jul. 1, 2004

(54) Wireless ECG system

(75) Inventors: Rud Istvan, Fort Lauderdale, FL (US); Bill Gregory, Fort Lauderdale, FL (US); Kenneth Solovay, Weston, FL (US); David Paul Chastain, Acton, MA (US); John David Gundlach, Acton, MA (US); Nicholas C. Hopman, Lake Zurich, IL (US); Daniel L. Williams, Norwell, MA (US); Franco Lodato, Weston, FL (US); Michael Salem, Ft. Lauderdale, FL (US)

Correspondence Address: BANNER & WITCOFF, LTD., TEN SOUTH WACKER DRIVE, SUITE 3000, CHICAGO, IL, 60606, US

(73) Assignee: GMP Companies, Inc., Fort Lauderdale, FL

(21) Appl. No.: 10/349,486

(22) Filed: Jan. 22, 2003

Related U.S. Application Data
(63) Continuation-in-part of application No. 09/998,733, filed on Nov. 30, 2001.
(60) Provisional application No. 60/350,840, filed on Jan. 22, 2002.

(51) Int. Cl. A61B 5/04; A61N 1/08

(52) U.S. Cl. 600/509; 607/60

(57) ABSTRACT

A system for detecting physiological data from a patient and, more particularly, a system for detecting electrocardiograph (ECG) information from a patient and transmitting the information to a central monitoring station via telemetry.

### RELATED APPLICATIONS

The application is a continuation-in-part of and claims the benefit of the filing date pursuant to 35 U.S.C. § 120 of application Ser. No. 09/998,733, for a WIRELESS ECG SYSTEM, filed Nov. 30, 2001 (Attorney Docket No. 05123.00004), the disclosure and content of which is hereby incorporated by reference in its entirety. In addition, the application claims priority to provisional U.S. Application Serial No. 60/350,840, which was filed on Jan. 22, 2002, the disclosure and content of which is hereby incorporated by reference in its entirety.

### FIELD OF THE INVENTION

The present invention relates to a cardiac monitoring system and, more particularly, to a wireless electrocardiograph (ECG) system. The cardiac monitoring system of the present invention detects physiological data from a patient, such as electrocardiograph (ECG) information, and transmits the information to a central monitoring station via telemetry.

### BACKGROUND OF THE INVENTION

An electrocardiograph (ECG) system monitors heart electrical activity in a patient. Conventional ECG systems utilize conductive pads or electrodes placed on a patient in specific locations to detect electrical impulses generated by the heart during each beat. In response to detection of the electrical impulses from the heart, the electrodes produce electrical signals indicative of the heart activity. Typically, these electrical signals are directly transferred from the electrodes to a stationary ECG monitor via multiple cables or wires. The ECG monitor performs various signal processing and computational operations to convert the raw electrical signals into meaningful information that can be displayed on a monitor or printed out for review by a physician.

Telemetry systems provide an alternative to conventional hardwired ECG systems that require multiple cables and wires that ordinarily tether an ECG patent to an ECG monitor. Conventional telemetry systems utilize portable telemetry boxes, which are hardwired to multiple electrodes positioned on the patient's body. Electrical signals from the patient's heart are detected by the electrodes and collected by the telemetry box. In turn, the telemetry box processes the electrical signals into waveform data and transmits the data a modest distance to a drop antenna that is hardwired to a central monitoring station. The data received by the drop antenna is transmitted to the central monitoring station where health care practitioners can remotely view and monitor the real time electrocardiograph data of the patients connected to the telemetry system.

To use existing telemetry systems, however, hospitals must retrofit their wards with an extensive network of cables and antennas to relay the information from the patient to the central monitoring station. The cost associated with the cables, antennas, and installation of the system is significant. In addition, many of the existing telemetry systems are proprietary and are not designed to operate with conventional stationary ECG monitors or other telemetry components. Thus, a need exists for an ECG telemetry system that is cost effective and universally compatible with existing or conventional telemetry systems and ECG components.

### BRIEF SUMMARY OF THE INVENTION

The present invention relates to a wireless ECG system that is universally compatible with existing or conventional ECG monitors. In addition, the present invention relates to an electrocardiograph telemetry system for collecting and transmitting electrocardiograph information and other physiological data from a patient and transmitting the information and data to a central monitoring station via telemetry. The present invention includes a data collection unit for collecting the physiological data from a patient. The data collection unit comprises a chest assembly and a remote electronics unit. The chest assembly is positioned on the patient and detects electrical signals of a patient's heart. The chest assembly connects to the remote electronics unit and transmits the electrical signals to the remote electronics unit. The remote electronics unit processes the signals and transmits the data to a repeater.

The repeater is capable of receiving and relaying data transmissions from multiple remote electronics units simultaneously. Multiple repeaters are positioned in locations throughout the hospital to provide cell pattern coverage consisting of overlapping zones so that each patient using the system will be within the range of multiple repeaters at any given time. Each repeater, in turn, relays the transmissions from the remote electronics unit to a central monitoring station. The central monitoring station includes a central base station that processes signals sent from multiple repeaters and transmits the data to a monitor or plurality of monitors where hospital personnel can remotely view and otherwise monitor the real time physiological data of the patients connected to the system.

These as well as other novel advantages, details, embodiments, features, and objects of the present invention will be apparent to those skilled in the art from the following detailed description of the invention, the attached claims and accompanying drawings, listed herein below which are useful in explaining the invention.

### BRIEF DESCRIPTION OF THE DRAWING

The foregoing aspects and many of the advantages of the present invention will become readily appreciated by reference to the following detailed description of the preferred embodiment, when taken in conjunction with the accompanying drawings, wherein:

FIG. 1 is a perspective view of an exemplary embodiment of the ECG system;

FIG. 2 is a cross sectional view of the chest assembly and the precordial assembly;

FIG. 3 is a top view of an exemplary embodiment of the chest assembly;

FIG. 4 is a top view of an exemplary embodiment of the precordial assembly;

FIG. 5 is a perspective view of an exemplary embodiment of the body electronics unit;

FIG. 6 is a top view an exemplary embodiment of the assembly connectors;

FIG. 7 is a front view of an exemplary embodiment of the body electronics unit;

FIG. 7a is an exemplary embodiment of the user interface of the electronics body unit;

FIG. 8 is a block diagram of an exemplary embodiment of the transmitter;

FIG. 9a is a perspective view of an exemplary embodiment of the base station used in conjunction with the token key;

FIG. 9b depicts the body electronics unit used in conjunction with the token key;

FIG. 10 is a perspective view of an exemplary embodiment of the base station;

FIG. 11 is a front view of an exemplary embodiment of the base station;

FIG. 11a is an exemplary embodiment of the user interface of the base station;

FIG. 12 is a block diagram of an exemplary embodiment of the receiver;

FIG. 13 is a perspective view of an exemplary embodiment of the base station;

FIG. 14 is a flow chart of an exemplary embodiment for operation of the ECG system; and

FIG. 15 depicts an exemplary embodiment of the telemetry system.

### DESCRIPTION OF THE PREFERRED EMBODIMENT

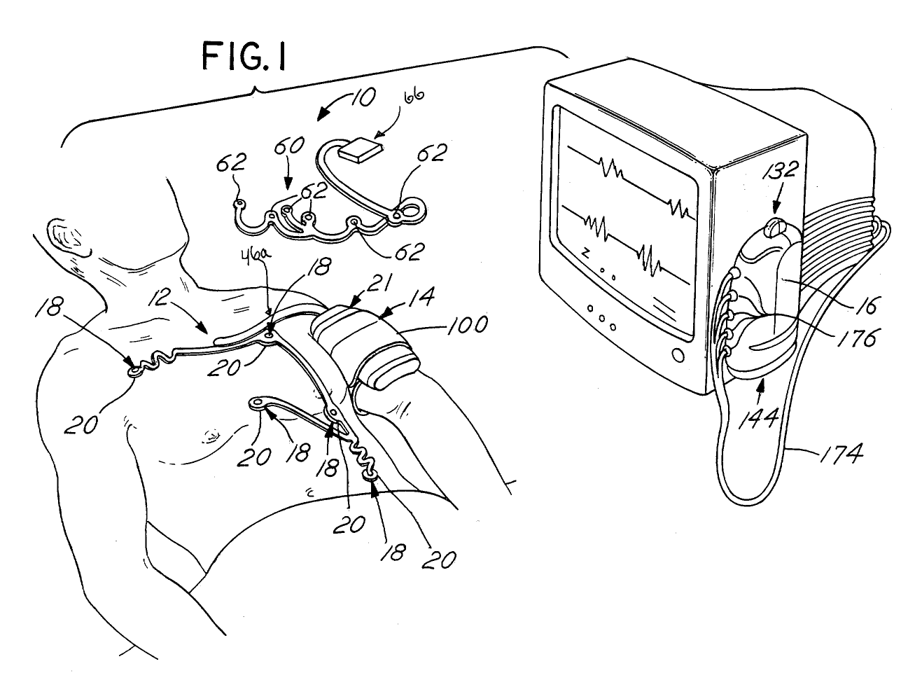

For a better understanding of the present invention, reference may be had to the following detailed description taken in conjunction with the appended claims and accompanying drawings. Briefly, the present invention relates to a wireless, portable ECG system. Referring to FIG. 1, the ECG system 10 comprises a chest assembly 12, a body electronics unit 14, and a base station 16.

The chest assembly 12 is a one-piece flexible circuit that connects a plurality of electrode connectors 18. Referring to FIG. 3, the electrode connectors are individually labeled 18a, 18b, 18c 18d, and 18e. The electrode connectors 18 have releasable connections that connect to electrodes or sensors 20. Preferably, the electrode connectors 18 have snap terminals that connect to electrodes 20 having snap terminals. Each electrode connector 18 connects to an electrically conductive element or trace for transmitting electrical signals. The electrically conductive elements or traces run along the chest assembly 12 and connect to a chest assembly connector 21.

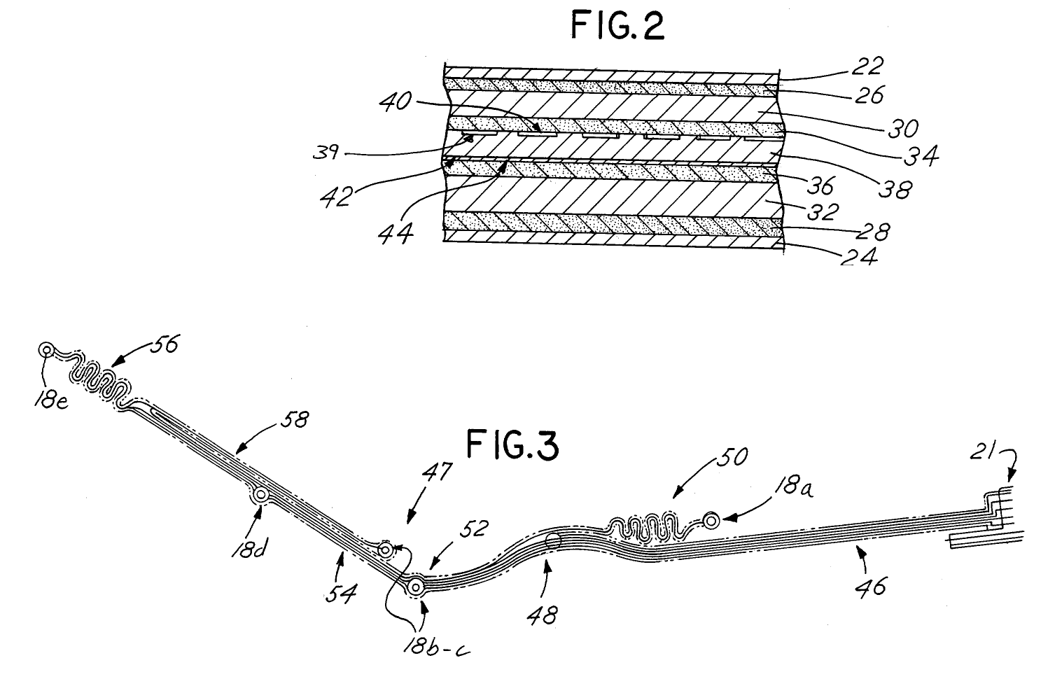

Referring to FIG. 2, the chest assembly 12 has outer layers 22, 24 that are constructed of a lightweight and reasonably moisture resistant material, such as DuPont Sontara® or other suitable fabric. Adhesive layers 26, 28 secure insulating layers 30, 32 to the outer layers 22, 24 respectively. Insulating layers 30, 32 are constructed of Mylar® (polyester) film or other suitable insulating material. Adhesive layers 34, 36 secure the insulating layers 30, 32 to a base layer 38. The base layer 38 is preferably constructed of Mylar film and has a first side 40 and a second side 42. The electrically conductive elements or traces that connect to the electrode connectors 18 are located on the first side 40 of the base layer 38. One such conductive element or trace is shown at 39. A shielding layer 44 for reducing any external inferences or radio frequency noise with the chest assembly 12 is located on the second side 42 of the base layer 38. The shielding layer 44 may be constructed of single or multiple layers of dielectric, or electrically or magnetically conductive material. The back of the electrode connector 18 may also be covered with Mylar to further insulate the chest assembly 12 and prevent an externally applied electric potential from entering the ECG system. The shielding layer preferably comprises an X-patterned grid.

Referring back to FIG. 1, the chest assembly 12 attaches to five electrodes 20 and provides a means for generally positioning the electrodes on the patient, thereby providing up to a “7 lead” analysis of the electrical activity of the heart. The electrode connectors 18 are preferably labeled and color-coded to ensure that the chest assembly 12 is properly positioned on the patient and connected to the appropriate electrodes or sensors 20. For instance, referring back to FIG. 3, the electrode connectors 18a, 18b, 18c, 18d, 18e are labeled RL, LA, LL, RA, and V, respectively. The chest assembly 12 is constructed such that the RA electrode connector is connected to an electrode positioned on the right side of the patient's chest about level of the first and second intercostal space, the LA electrode connector is connected to an electrode positioned on the left side of the patient's chest about level of the first and second intercostal space, the RL and LL electrode connectors are connected to electrodes positioned on the left side of the patient's torso, and the V electrode connector is connected to an electrode positioned in the middle of the patient's chest about level of the fourth and fifth intercostal space. The chest assembly 12 is designed such that it is centered on the chest below the patient's clavicle.

Referring to FIG. 3, the chest assembly 12 is configured to provide flexible positioning of the chest assembly 12 on the patient. FIG. 3 is for illustrative purposes only, and thus, the chest assembly 12, as depicted in FIG. 3, is not limited to any particular shape or configuration. The chest assembly 12 has a linear section or tail 46 extending from the chest assembly connector 21. Referring back to FIG. 1, the tail 46 has a securing means 46a that allows the tail 46 to extend to either side of the patient. This securing means 46a may be any suitable mechanical device although adhesive or a clip is most preferred. Referring back to FIG. 3, the tail 46 flows into an electrode retaining section 47. The electrode retaining section 47 has an arcuate section 48. A first expandable arm 50 attaches to the arcuate section 48. The RA electrode connector attaches to the first expandable arm 50. The arcuate section 48 flows into a transition section 52. The LA electrode connector attaches to the transition section 52. The transition section 52 flows into a linear run 54. The RL electrode connector attaches to the linear run 54. A second expandable arm 56 and an extension arm 58 attach to the linear run 54. The V electrode connector attaches to the second extension arm 58 and the LL electrode connector attaches to the second expandable arm 56.

The expandable arms 50, 56 are die cut in a serpentine pattern. The expandable arms 50, 56 comprise polypropylene or polyethylene fabric, Kapton, Mylar, or other flexible, memory-less material. The expandable arms 50, 56 expand, if necessary, by elongating the serpentine pattern. When expanded, a portion or all of the expandable arm is extended. Where only a portion of the expandable arm is extended, another portion remains folded. The expandable arms 50, 56 allow for extension as needed to so that the chest assembly 12 can fit patients of various sizes and also allow for patient movement when the patient is wearing the chest assembly 12. The extension arm 58 allows for flexible positioning of the V electrode connector in the middle of the patient's chest such as placement at electrode position V1, V2 or V3. In some instances, the health care practitioner may desire not to utilize the extension arm 58 for taking electrocardiograph measurements. Thus, to keep the extension arm 58 secured to the linear run 54 and to ensure that the extension arm 58 will not interfere with the placement and positioning of the chest assembly 12, the extension arm 58 is die cut with a perforated seam that connects the extension arm 58 and the linear run 54 along the length of the extension arm 58. If the health care practitioner desires to use the extension arm 58, the perforated seam is unbroken so that the extension arm 58 can be selectively positioned on the patient's chest.

The chest assembly 12 can be used with a precordial assembly 60 to provide a “12-lead” analysis of the electrical activity of the heart. Similar to the chest assembly 12, the precordial assembly 60 is a one-piece flexible circuit that connects a plurality of electrode connectors 62. The electrode connectors 62 have releasable connections that connect to electrodes (not shown). Preferably, the electrode connectors 62 have snap terminals that connect to electrodes having snap terminals. Each electrode connector 62 connects to an electrically conductive element or trace for transmitting electrical signals from a patient's heart. The electrically conductive elements or traces run along the precordial assembly 60 and connect to a precordial assembly connector 66. The precordial assembly 60 has the construction as shown in FIG. 2.

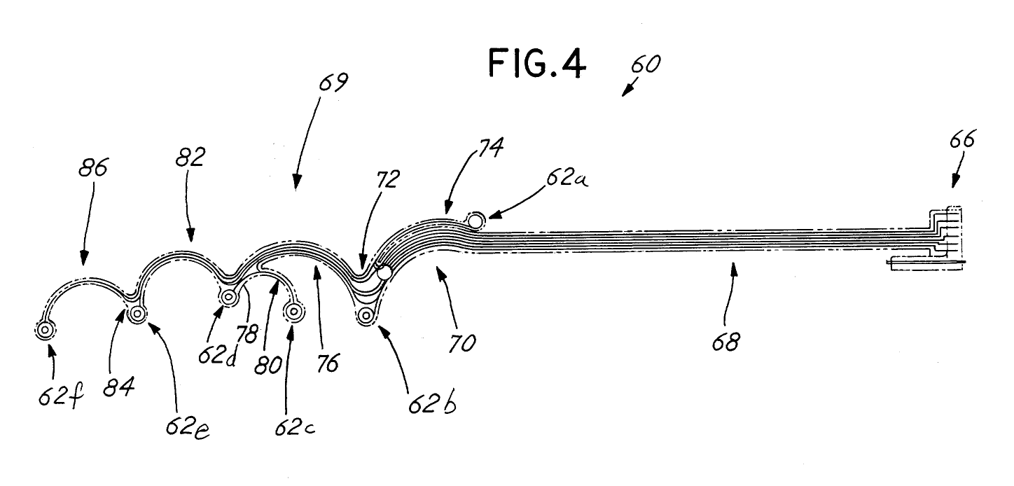

The precordial assembly 60 can attach to six electrodes that are selectively positioned on the abdomen and middle chest of the patient. The electrode connectors 62 of the precordial assembly 60 are preferably labeled and color-coded so as to prevent a health care provider from applying or positioning the precordial assembly onto the patient improperly. For instance, as referring to FIG. 4, the electrode connectors 62a, 62b, 62c, 62d, 62e, and 62f are labeled V1, V2, V3, V4, V5, and V6, respectively. When the precordial assembly 60 is used, the V electrode connector on the chest assembly 12 is removed from its electrode and replaced with an electrode connector on the precordial assembly 60.

As shown in FIG. 4, the precordial assembly 60 is configured to provide flexible positioning of the precordial assembly 60 on the patient. FIG. 4 is for illustrative purposes only, and thus, the precordial assembly 60, as depicted in FIG. 4, is not limited to any particular shape or configuration. The precordial assembly has a linear section or tail 68 extending from the precordial assembly connector 66. The linear section or tail 68 flows into an electrode retaining section 69. The electrode retaining section 69 has a first arcuate section 70 having a first transition section 72. The V2 electrode connector attaches to the first transition section 72. The V1 electrode connector attaches to a first extension arm 74 connected to the first transition section 72. A second arcuate section 76 extends from the first transition section 72. A second transition section 78 abuts the second arcuate section 76 and the V4 electrode connector attaches to the second transition section 76. The V3 electrode connector attaches to a second extension arm 80 connected the second transition section 78. A third arcuate section 82 flows from the second transition section 78. The third arcuate section 82 abuts a third transition section 84. The V5 electrode connector attaches to the third transition section 84. A fourth arcuate section 86 extends from the third transition section 84. The V6 electrode attaches to the fourth arcuate section 86. The configuration of the precordial assembly 60 allows the health care provider or physician to flexibly position the electrode connectors 62 as needed to properly situate the precordial assembly 60 on the patient and to allow for patient movement when the patient is wearing the precordial assembly 60

In operation, the chest assembly 12 and the precordial assembly 60 detect electrical signals generated by the heart during each beat and transfer these signals to the body electronics unit 14. When the system is operating in “7 lead” mode (i.e. when only the chest assembly 12 is being used) the body electronics unit 14 acquires signals from the RL, RA, LL, LA, and V electrodes. The body electronics unit 14 uses the RL electrode as a ground reference. When the system is operating in the “12 lead” mode (i.e. the chest assembly 12 and the precordial assembly 60 are being used) the body electronics unit 14 acquires signals from the RL, RA, LL, and LA electrodes via the chest assembly 12 and acquires signals from the V1, V2, V3, V4, V5, and V6 electrodes via the precordial assembly 60. Alternatively, a various number of electrodes may be monitored by the system. For example, the health care provider or physician may choose to use only two electrodes to monitor the heart, seven electrodes to monitor the heart, or the like. In other words, the present system is not limited to performing a “7 lead” and “12 lead” analysis of the heart. In addition, to detecting electrical signals from the heart, the chest assembly 12 and the precordial assembly 60 may be constructed to detect other vital signs of the patient, for example, pulse, respiration rate, heart rate, temperature EEG signals, and pulse oximeter signals.

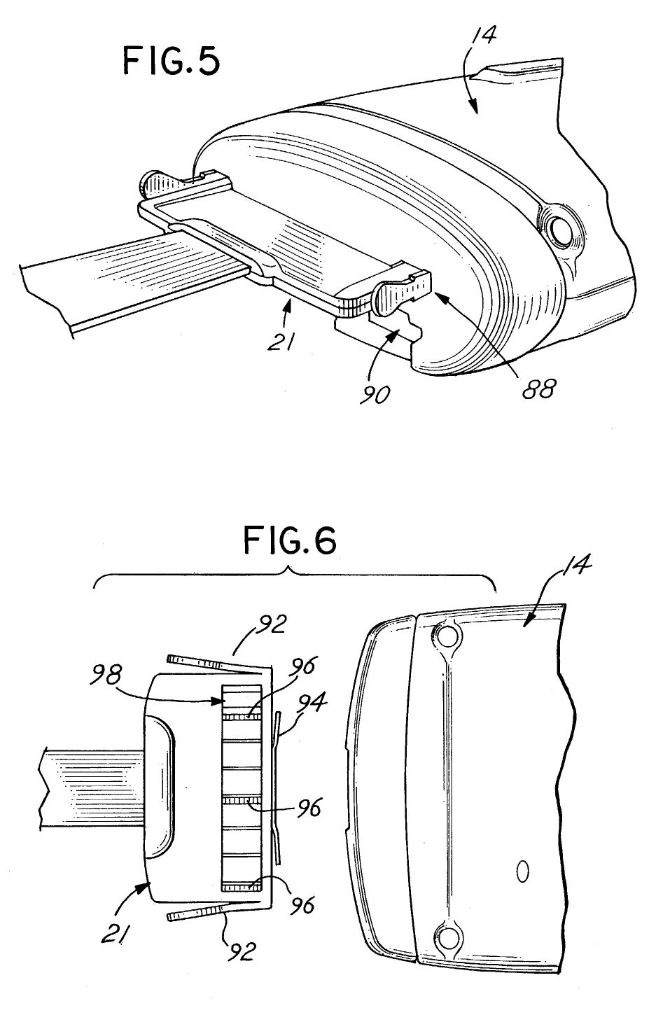

Referring to FIG. 5, the chest assembly 12 connects to the body electronics unit 14 via a chest assembly connector 21. Specifically, the chest assembly connector 21 inserts into a chest assembly port 88 located in the body electronics unit 14. Similarly, the precordial assembly 60 connects to the body electronics unit 14 via the precordial assembly connector 66. Specifically, the precordial assembly connector 66 (not shown) inserts into a precordial assembly port 90. Resisters are connected to the chest assembly port 88 and the precordial assembly port 90 to prevent excessive electrical current from entering the body electronics unit 14—thereby ensuring that the body electronics unit 14 continues to operate properly in the presence a strong electrical current caused by a defibrillator (i.e. a 5 kV defibrillation excitation). The chest assembly connector 21 and the precordial assembly connector 66 are specifically keyed or configured to prevent the assembly connectors 21, 66 from being inserted into the assembly ports 88, 90 backwards, misaligned or otherwise improperly. Moreover, the chest assembly connector 21 is keyed or configured such that it is not compatible with the precordial assembly port 90. Likewise, the precordial assembly connector 66 is keyed or configured such that it is not compatible with the chest assembly port 88. Specifically, the chest assembly connector 21 has tongues specifically configured or arranged to fit into corresponding grooves of the chest assembly port 88. Accordingly, the chest assembly connector 21 can only be connected to the chest assembly port 88 in one orientation. For example, if the tongues are not aligned with the grooves, the chest assembly connector 21 will not couple to the chest assembly port 88. Likewise, the precordial assembly connector 66 has tongues (not shown) specifically configured or arranged to fit into corresponding grooves (not shown) of the precordial assembly port 90.

As shown in FIG. 6, the chest assembly connector 21 and the precordial assembly connector 66 (not shown) have retaining clips or flanges 92 located on the sides of the connectors 21, 66 for removably securing the connectors 21, 66 into the assembly ports 88, 90. However, other means may be used to removably secure the connectors 21, 66 in the assembly ports 88, 90, such as screws, pins or the like. In addition, the assembly connectors 21, 66 may have spring flanges or clips 94 located at the tip of the connectors 21, 66 for providing a bias or tension against the assembly ports 88, 90. The spring flanges or clips 94 provide the connectors 21, 66 with a secure fit within the assembly ports 88, 90, thereby reducing any play or movement of the connectors 21, 66 within the assembly ports 88, 90. The electrically conductive elements or traces are specifically configured on the connectors 21, 66 so as to ensure that the electrical signals from the heart are properly transmitted to the body electronics unit 14. In other words, the electrically conductive elements or traces must be sufficiently spaced apart or otherwise isolated in some manner to prevent arcing across the electrically conductive elements. In addition, the spacing of the electrically conductive elements or traces permits the chest assembly and the precordial assembly to withstand defibrillation shock. Furthermore, the connectors 21, 66 have ribs 96 for preventing the electrically conductive elements or traces from coming into contact with metal objects or the like when the connectors 21, 66 are not inserted into the assembly ports 88, 90.

The chest assembly connector 21 has a sensor pin or ground pin 98 that completes a circuit within the body electronics unit 14 when the chest assembly connector 21 is plugged into the chest assembly port 88, thereby activating the power and bringing the body electronic unit 14 out of “sleep mode.” The sensor pin has specific tongue that corresponds and fits into a groove located in the chest assembly port 88. The sensor pin 98 serves as a means for the body electronics unit 14 to identify the chest assembly 12 and to prevent the use of other chest assemblies or electrocardiograph wearables that are not designed to be used with the on-body electronic unit 14. In other words, the power of the body electronics unit 14 will not activate unless the body electronics unit 14 identifies or recognizes the sensor pin 98 of the chest assembly 12.

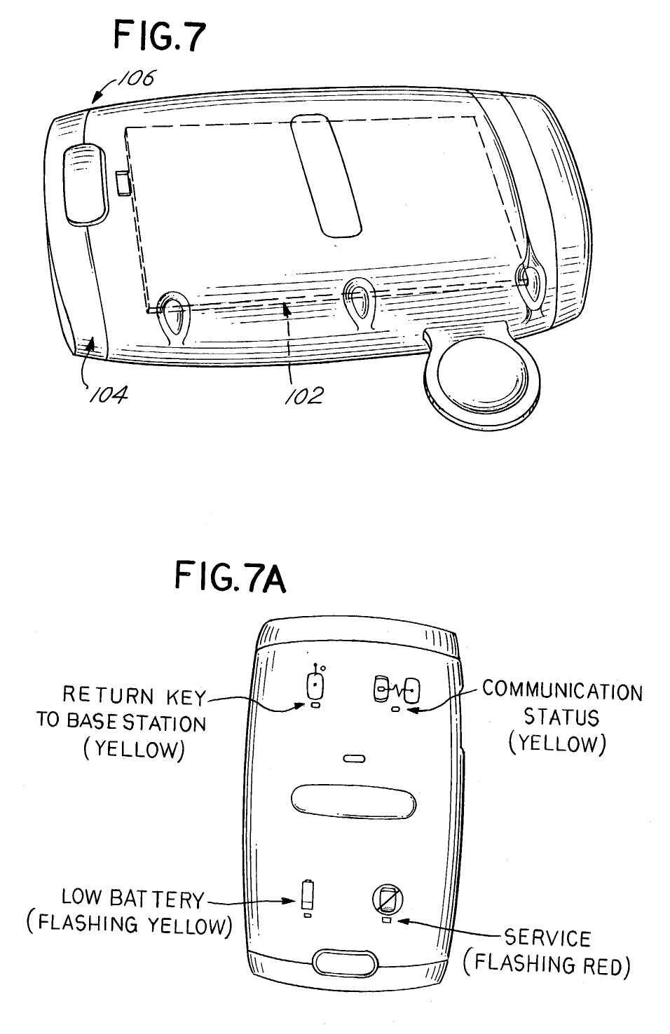

The outside casing of the body electronics unit 14 is constructed of lightweight, molded plastic, such as acrylonitrile-butadiene-styrene (ABS) or other suitable material. The shape and configuration of the body electronics 14 unit is not limited to any particular shape or configuration. As shown FIG. 1, the body electronic unit 14 removably secures to the patient's arm via an armband 100, thus making the body electronics unit 14 readily accessibly to the patient. The armband 100 wraps around either the patient's right or left arm and attaches via Velcro or other suitable fastening means such as pins, snaps, or the like. The body electronics unit 14 slides under a strap or pocket on the armband 100. Referring to FIG. 7, the body electronic unit 14 has a user interface 102 and a battery 104. The user interface 102 provides information to the patient pertaining to the system's operating status or functionality. For example, an exemplary embodiment of the user interface 102 may provide information on whether the body electronics unit 14 is communicating or transmitting normally to the base station 16, whether the battery 104 of the body electronics unit 14 is charging or the battery 104 is low, whether the power of the body electronics unit 12 is activated, or whether the body electronics unit 14 or base station is malfunctioning. In addition the user interface 102 may provide instructions on the correct order or procedure for pairing or coupling the body electronics unit 14 with the base station 16. Such information may be communicated to the patient via the user interface 102 in various ways, for example, LEDs, LCD, text, audible tones, etc. An exemplary embodiment of the user interface is shown in FIG. 7a. The user interface 102 is readily accessible to the patient when the body electronics unit 14 is secured to the armband 100.

The battery 104 is inserted into a battery port 106 located in the bottom of the body electronics unit 14. The battery 104 is retained in the battery port 106 by latches or other suitable fastening means, such as clips, screws or the like. The battery 104 is preferably a 3.6 V Li-ion rechargeable battery. The battery 104 is readily accessible to the patient when the body electronics unit 14 is secured to the armband 100.

The body electronics unit 14 controls the acquisition of the ECG signals from the chest assembly 12 and the precordial assembly 60. A transmitter within the body electronics unit 14 receives or acquires ECG signals from the chest assembly 12 and the precordial assembly 60 preferably at 3 kbps. When the system is operating in “7 lead” mode (i.e. when only the chest assembly 12 is being used) the body electronics unit 14 acquires signals from the RL, RA, LL, LA, and V electrodes. When the system is operating in the “12 lead mode” (i.e. the chest assembly 12 and the precordial assembly 60 are being used) the body electronics unit 14 acquires signals from the RL, RA, LL, and LA electrodes via the chest assembly 12 and acquires signals from the V1 thru V6 electrodes via the precordial assembly 60. In addition, other vital signs of the patient may be detected by the system and transmitted to the body electronics unit 14, for example pulse, respiration rate, heart rate, temperature, EEG signals and pulse oximeter signals. No waveform processing of the physiological data collected from the patient is conducted in the body electronics unit 14. Instead, all waveform processing of the signal is either performed at the base station 16 or a conventional monitor. In contrast, in conventional telemetry systems, the waveform processing of the physiological data is performed in the remote electronics or telemetry unit.

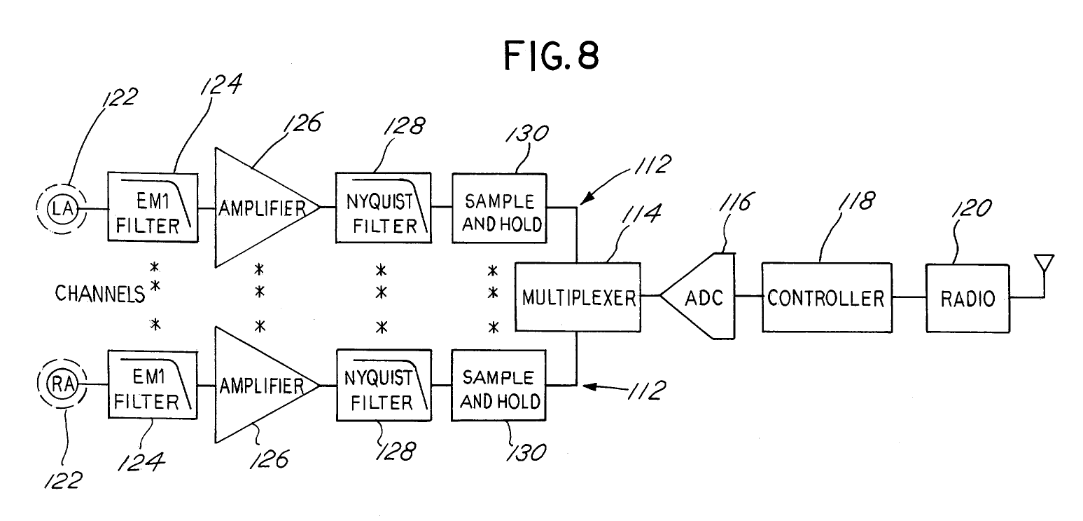

Referring to FIG. 8, the transmitter comprises an application specific integrated circuit, a processor or other circuit a plurality of signal channels 112, a multiplexer 114, an analog-to digital converter (ADC) 116, a controller 118, and a radio 120. Additionally, fewer or different components can be used. The body electronics unit 14 has nine signal channels 112 corresponding to the ten electrodes connected to the chest assembly 12 and the precordial assembly 60. The electrode channels 112 each comprise a connector 122, a filter 124, an amplifier 126, a Nyquist filter 128 and a sample and hold circuit 130. The connectors 122 of the signal channels 112 connect to either the chest assembly port 88 or the precordial assembly port 90, depending on whether the electrode channel 112 corresponds to an electrode located on the chest assembly 12 or the precordial assembly 60. The filter 124 comprises a low pass filter, such as for removing electromagnetic interference signals. The amplifier 126 amplifies the signals from the electrodes. The Nyquist filter 128 comprises a low pass filter for removing out-of-band high frequency content of the amplified signals to avoid sampling error. The sample and hold circuit 130 enables the system to sample all nine electrode channels signals 112 at a same or relative times so that there is no differential error created when these signals are combined later in an ECG monitor.

The multiplexer 114 sequentially selects signals from the electrode signal channels 112 using time division multiplexing. One of ordinary skill in the art, however, recognizes that other combination functions can be used. The ADC 116 converts the combined analog signals to digital signals for transmission. Preferably the controller 118 comprises a digital signal processor (DSP) that decimates the digitized signals as to lessen the bandwidth required to transmit the signals. The radio 120 modulates the digital signals with a carrier signal for transmission. In an exemplary embodiment, the radio 120 includes a demodulator for receiving information. The controller 118 digitally transmits the ECG data to the base station 16. In an alternative embodiment, the controller 118 transmits the ECG data to a repeater that may be located in various locations throughout a hospital (described in detail below). In addition to transmitting ECG data, the controller 118 may transmit signals pertaining to pacemaker information, battery level information, electrode disconnection information, and other information as required. For example, vital signs such as pulse, respiration rate, heart rate, temperature, EEG signals, and pulse oximeter signals may be transmitted.

The body electronics unit continuously monitors the integrity of all patient electrode connections. In the event a lead is disconnected, the body electronics unit will send a signal to the base station, or a repeater and then to a base station, that in turn causes the base station to trigger the “lead off” alarm on the ECG monitor. Additionally, the body electronics unit has a self-test function that monitors the integrity of the primary functions including the microprocessor, data acquisition, internal voltage references, and radio functionality. In the event a failure is detected, the body electronics unit will capture the fault condition, stop data acquisition and transmission and indicate that fault has occurred through the lead off alarm.

The body electronics unit 14 operates to minimize undesired noise or signals. For example, components are matched such that later application to a differential amplifier in a legacy ECG monitor for determining a heart vector is accurate. ECG vectors are not formed by the ECG system 10, but rather by the legacy ECG monitor. Because the ECG system 10 is essentially “in-series” with the legacy ECG monitor, any error may produce undesirable results. One potential source of error is differential error. This differential error can be observed on the legacy ECG monitor when the ECG monitor forms the ECG lead signals by combining the individual electrode signals in the ECG monitor input stage. This input stage comprises a difference, or differential, amplifier to eliminate common mode interference from the signals produced at the electrodes 20.

An artifact will be present if there is any difference in how each of the electrode signals are processed when the legacy ECG's differential amplifier forms the ECG lead signals or ECG vectors. For example, if there is a difference in the gain of the amplifier, a difference in the phase shift associated with the anti-aliasing (Nyquist) filters, or a difference in how the respective track and hold circuits treat the electrode signals, then this differential error creates an artifact on the legacy ECG monitor. One important technique to minimize this potential source of differential errors is to choose a Nyquist filter cutoff frequency that is very high. This is because each individual filter will have differing group delay performance. To mitigate that difference, the frequency that this group delay will affect is much higher than the frequency of the ECG signals, which are about 0.05 Hz to 150 Hz. By choosing a high cutoff frequency for the Nyquist filters, any mismatch in the Nyquist filter components will not affect accuracy of the individual electrode ECG signals. For example, picking a filter cutoff frequency of 1,200 Hz mitigates this source of error. With this approach, the individual electrode ECG signals are over sampled at about 3,000 Hz in order to not introduce aliasing. Of course higher filter cutoff frequencies and correspondingly higher sampling rates may further reduce error. Lower cutoff frequencies and/or sampling rate may be used.

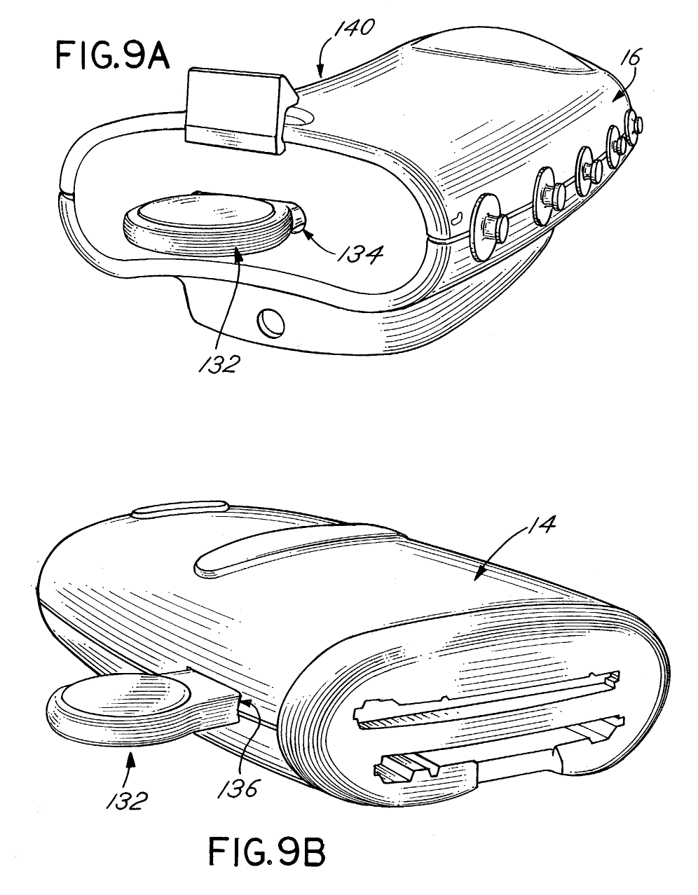

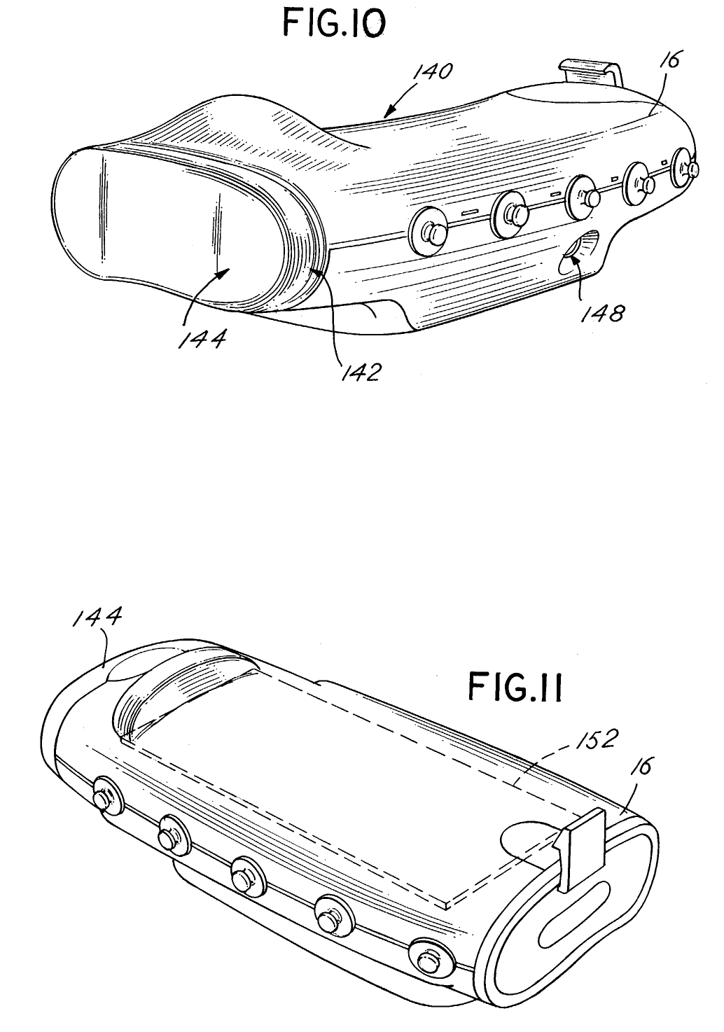

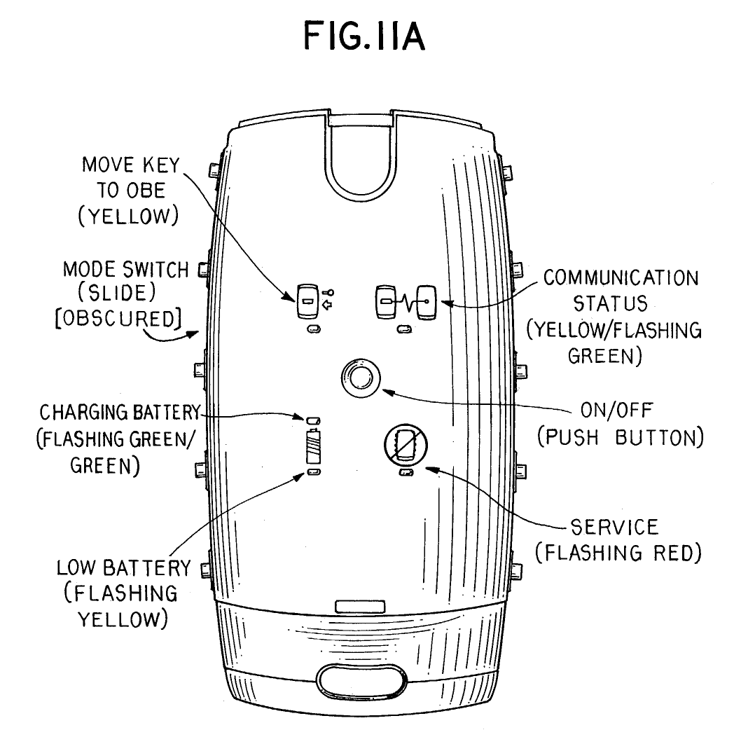

Because the electrode signals are now sampled at such a high rate, these signals may be decimated to minimize the required transmission bandwidth. For example the digital samples are decimated by a factor of eight in the controller 118. Greater or lesser rates of decimation can be used, such as decimation as a function of the bandwidth available for transmission, the number of electrode signals to be represented, and the Nyquist sampling rate. Referring back to FIG. 1, the base station 16 receives the transmitted signals sent from the body electronics unit 14. The signals are transmitted as radio or other signals modulated with a carrier signal. Various air-interfaces can be used for transmission, such as Bluetooth or IEEE 802.11b. To establish proper communication between the body electronics unit 14 and the base station 16, the base station 16 and body electronics unit 14 need to be paired such that the base station 16 and the body electronics unit 14 only recognize signals from the its pair. This may be accomplished in number of ways including direct connection of the base station 16 and the body electronics unit 14. Preferably, a token key 132 is used to pair or radio frequency link the body electronics unit 14 and the base station 16. Referring to FIG. 9a, the token key 132 has memory chip and may optionally have a plurality of tongues or pins that fit within grooves located in a token key port 134 of the base station 16 and within grooves of a token key port 136 of the body electronics unit 14. As shown in FIG. 9b, the token key 132 inserts into the token key port 134 of the base station and reads and records an identification number for the base station 16. The token key 132 is then removed from the token key port 134 and inserted into the token key port 136 located in the body electronics unit 14. The electronics unit 14 receives the identification number for the base station 16 from the token key 132. In turn, the token key 132 reads and records the identification number for the body electronics unit 14. The token key 132 is then removed from the body electronics unit 14 and reinserted into the token key port 134 of the base station 16 whereby the base station 16 confirms the presence of its own identification number on the token key 132 and also reads the identification number for the body electronics unit 14 from the token key 132. The body electronics unit 14 and the base station 16 are paired. Alternatively, pairing or coupling can be accomplished by first inserting the token key 132 into the body electronics unit 14, removing the token key 132 and inserting the token key 132 into the base station 16, removing the token key 132 and reinserting the token 132 into the body electronics unit 14. In other words, the order in which the token key 132 is inserted into the body electronics unit 14 and the base station 16 is not critical to the proper operation of the system. Referring back to FIG. 7, the user interface 102 may provide the user or health care provider with instructions on the correct order for pairing the body electronics unit 14 with the base station 16. The use of the token key 132 allows the pairing function to occur while the body electronics unit 14 is worn by the patient. This feature eliminates the need to disconnect and reconnect the body electronics unit 14 when a patient needs to be connected to different ECG monitors as a result of being moved around a hospital. The patient's body electronics unit 14 is just repaired with a new base station using the token key 132.

After the body electronics unit 14 and the base station 16 are paired, the body electronics unit 14 and the base station 16 will remain communicating with each other as long as the token key 132 remains in the token key port 134 of the base station 16 (or the token key port 136 of the body electronics unit 14, depending on the order of the pairing process). In other words, as soon as the token key 132 is removed from the base station 16, the electronics unit 14 and the base station 16 will discontinue or cease communication. Any specific token key 132 can be used to pair any specific base station 16 with any specific body electronics unit 14.

The outside casing of the base station 16 is constructed of lightweight, molded plastic, such as acrylonitrile-butadiene-styrene (ABS) or other suitable material. The shape and configuration of the base station 16 is not limited to any particular shape or configuration. As shown in FIG. 1, the base station 16 may be removably secured to an ECG monitor via suitable mounting means, such as Velcro®, dual-lock strips, double-sided foam tape, or the like. Preferably, the base station 16 is removably mounted to a mounting plate secured near the ECG monitor via suitable mounting means. As shown in FIG. 10, the base station 16 has a cradle 140 for storing the body electronics unit 14 when the body electronics unit 14 is not in use or otherwise off the patient. In addition, the base station 16 has a battery port 142 in which a base station battery 144 is removably inserted. The base station 16 may be constructed to have a plurality of battery ports that store and charge batteries when the batteries are not being used. When the base station 16 is not plugged into an AC wall power inlet, the base station battery 144 provides power to the base station 16. When the base station 16 is operating on AC wall power, the base station 16 charges the base station battery 144 when the base station battery 144 is in the battery port 142. The base station 16 has a power switch that activates/deactivates the power to the base station 16 and a power cord connection 148 for connecting a power cord to an AC wall power inlet. The base station battery 144 is preferably a 3.6 V Li-ion rechargeable battery. Accordingly, the base station battery 144 and the body electronics unit battery 104 are preferably identical and interchangeable, such that each battery can be used in either the body electronics unit 14 or the base station 16. The system is designed such that a discharged body electronics unit battery 104 is swapped for a charged base station battery 144. In this manner a charged battery is always readily available for the body electronics unit. In addition, the base station may have a lead switch that allows the health care provider to instruct the base station 16 to operate in “7 lead” mode or “12 lead” mode.

As depicted in FIG. 11, the base station 16 has a user interface 152 that provides information to the health provider or patient pertaining to the system's operating status or functionality. For example, the user interface 152 may provide information on whether the body electronics unit 14 is communicating or transmitting normally to the base station 16, whether the base station battery 144 is charging or the battery 144 is low, whether the body electronics unit battery 104 is low, or whether the power of the base station 16 is activated, whether the base station 16 is malfunctioning or otherwise requires servicing. In addition the user interface 102 may provide instructions on the correct order or procedure for pairing or coupling the body electronics unit 14 with the base station 16. Such information may be communicated to the health care provider or patient via the user interface 152 in various ways, for example, LED's, LCD, text, audible tones, etc. An exemplary embodiment of the user interface 102 is shown in FIG. 11a.

Additionally, the base station has a self-test function which monitors the integrity of the primary functions including the microprocessor, data acquisition, internal voltage references, and radio functionality. In the event a failure is detected, the body electronics unit will capture the fault condition, stop data acquisition and transmission and indicate that fault has occurred through the lead off alarm.

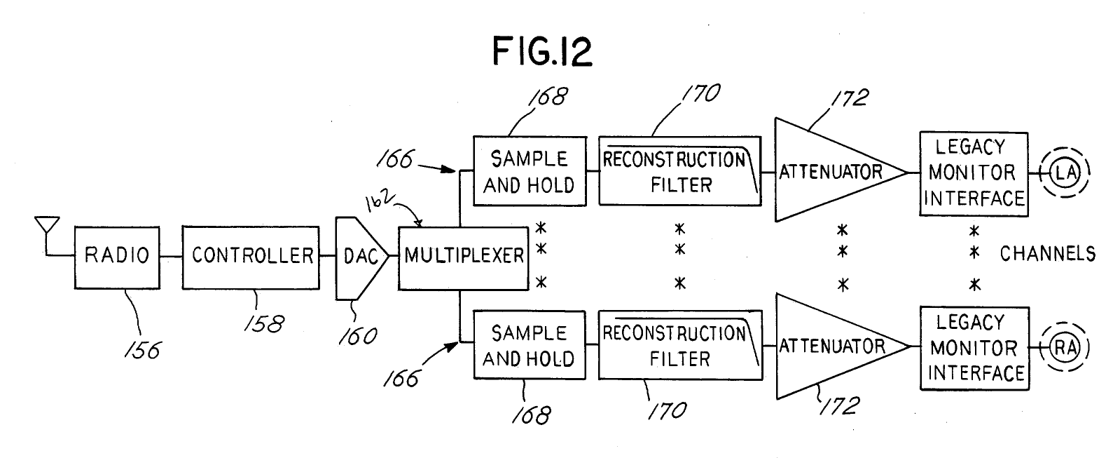

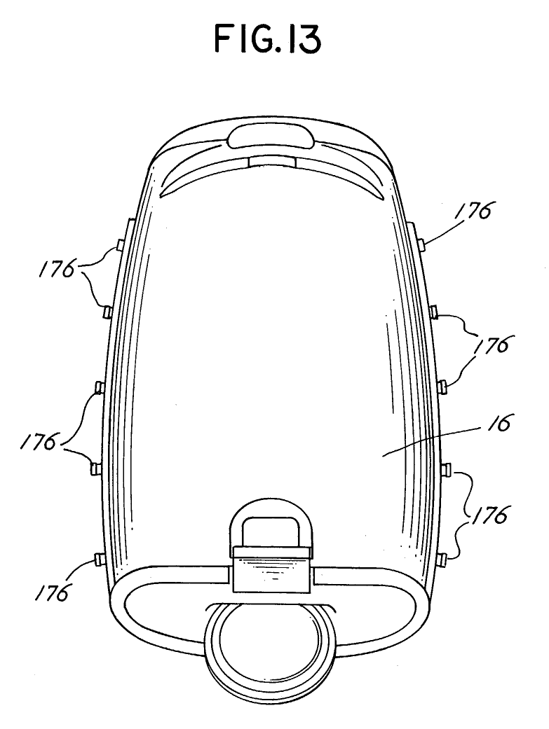

A receiver located within the base station 16 receives signals sent to the base station 16 from the body electronics unit 14. Referring to FIG. 12, the receiver may include a radio 156, a controller 158, a digital-to-analog converter (DAC) 160, a de-multiplexer 162, a transceiver, and a plurality of electrode signal channels 166. The radio 156 demodulates the received signals for identifying digital data representing the combined electrode signals. In an exemplary embodiment, the radio 156 includes a modulator for transmitting control information. The controller 158 controls operation of the various components and may further process the signals from the radio 156, such as interpolating data, converting the signals to digital information, generating control signals for the transmitter 108 in the electronics unit 14, operating any user output or input devices, and diagnosing operation of the ECG system. Preferably, the controller 158 interpolates the electrode signals to return the effective sample rate to about 3 kHz or another frequency. This enables the reconstruction filters to have a cutoff frequency many times the bandwidth of the electrode signals, thus minimizing any differences in group delay at the frequencies of interest, i.e. less than 150 Hz. The DAC 160 converts the digital signals to analog signals. The demultiplexer 162 separates the individual regenerated electrode signals onto the separate electrode signal channels 166. The transceiver 164 operates operable pursuant to the Bluetooth specification for two-way communication with the transmitter 108.

The receiver 154 has nine electrode signal channels 166 corresponding to the 10 electrodes connected to the chest assembly 12 and the precordial assembly 60. The electrode signal channels 166 each comprise a sample and hold circuit 168, a filter 170, and an attenuator 172. The sample and hold circuit 168 is controlled by the controller 158 so that the converted electrode signals appear simultaneously on each electrode signal channel 166. Other embodiments may include individual DAC's that provide the signal substantially simultaneously. The filter 170 comprises a low pass reconstruction filter for removing high frequency signals associated with the DAC conversion process. The attenuator 172 comprises an amplifier for decreasing the amplitude to a level associated with signals at the electrodes, which were earlier amplified in the amplifiers of the body electronics unit 14. This results in a unity system gain so as not to introduce error between the electrodes and the conventional ECG monitor.

The base station 16 transmits the ECG signals to the ECG monitor 138 via pre-existing or conventional monitor cables 174. In turn, the information is displayed on the ECG monitor and reviewed by a physician. As depicted in FIG. 13, the monitor cables 174 removably insert onto snap terminals 176 located on the base station 16. Preferably, the base station 16 has ten snap terminals 176 arranged on the left and right side of the base station 16. The snap terminals 176 and the monitor cables 174 are preferably labeled and color-coded so that the monitor cables 174 are properly connected to the base station 16. For instance, the five snap terminals 176 located on the left side of the base station 16 and the monitor cable 174 may be labeled as RL, LA, LL, RA, and V/V. In addition, the five snap terminals 176 on the right side of the base station 16 and the monitor cable 174 may be labeled V2, V3, V4, V5, and V6. When the ECG system is operating in “7 lead” mode (i.e. only the chest assembly 12 is used) the monitor cable 174 is plugged into the five snap terminals 176 on the left side of the base station 16. When the ECG system is operating in “12 lead” mode (i.e. both the chest assembly 12 and the precordial assembly 60 is used) both the monitor cables 174 are plugged into the snap terminals 176—the top four snap terminals 176 on the left side of the base station 16 will be used for chest assembly electrodes and the remaining six snap terminals 176 will be used for precordial assembly electrodes.

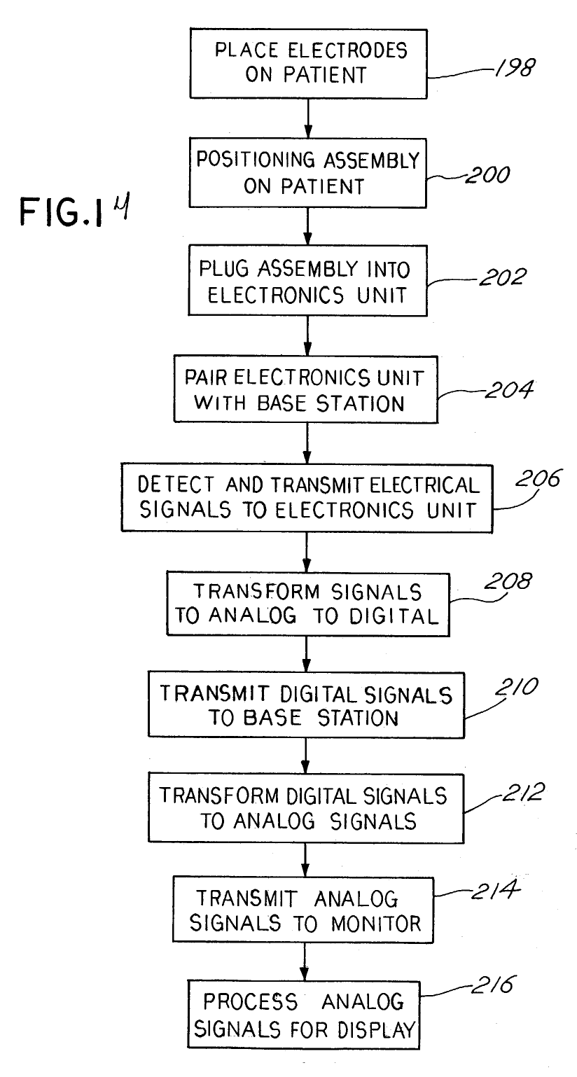

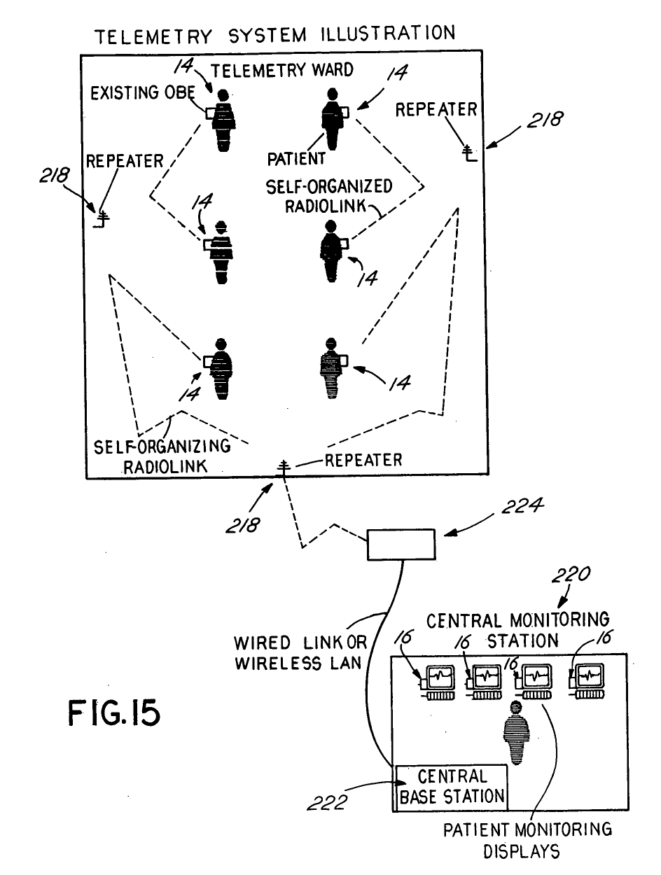

FIG. 14 depicts the method of monitoring the cardiac activity in the patient's heart using the wireless ECG system of the present invention. In step 198, electrodes are placed on the patient's body. In step 200, the chest assembly 12 and/or precordial assembly 60 are positioned on the patient's body by connecting the electrode connectors 21, 62 to the electrodes. In step 202, the chest assembly 12 and/or the precordial assembly 60 are plugged into the body electronics unit 14. In step 204, the electronics unit 14 and the base station 16 are paired or coupled by inserting the token key 132 into the base station 16, removing the token key 132 from the base station 16, inserting the inserting the token key 132 into the body electronics unit 14, removing the token key 132 from the electronics unit 14, and reinserting the token key 132 into the base station 16. Alternatively, coupling can be accomplished by inserting the token key 132 into the body electronics unit 14, removing the token key 132 from the body electronics unit, inserting the token key 132 into the base station 16, removing the token key 132 from the base station 16 and reinserting the token key 132 into the body electronics unit 14. In step 206, electrical signals from the patient's heart are detected and transmitted to the body electronics unit 14 via chest assembly 12 and the precordial assembly 60. In step 208, the electrical signals from the heart are transformed by the body electronics unit 14 from analog signals to digital signals. In step 210, the body electronics unit 14 transmits the digital signals to the base station 16 via radio transmission. In step 212, the base station 16 transforms the digital signals into analog signals. In step 214, the base station 16 transmits the analog signals to the ECG monitor 138 via monitor cables 174. In step 216, the ECG monitor 138 processes the analog signals into meaningful information that can be displayed on the monitor 138.

In an alternative embodiment, a body electronics unit 14 transmits the digital signals to a repeater 218, which relays the signal to the base station 16. As illustrated in FIG. 18, repeaters 218 may be located in various locations throughout a hospital as shown in FIG. 18. The body electronics unit 14 and the repeater 218 communicate via telemetry. Various air interfaces may be used to transmit the physiological data from the body electronics units 14 to the repeaters 218, for example Bluetooth, IEEE 802.11b, WiFi, or other suitable wireless LAN system protocols. The body electronics unit 14 may tag each digital signal sent to the repeater 218 with an electronic identification number that corresponds to the body electronics unit 14. As a result, if necessary, each signal can be traced back to the body electronics unit 14 in which it originated from.

Each repeater 218 is capable of communicating with a plurality of body electronics units 14. Each body electronics unit 14 may be configured with a “switch over” protocol in the which the body electronics unit 14 continuously attempts to establish connections with repeaters 218 as a patient moves throughout the hospital ward. This “switch over” protocol allows the body electronics unit 14 to transmit to those repeaters 218 that offer the best link performance or best signal strength given the patient's location within the hospital ward.

The repeaters 218 are spaced apart from each other and located throughout a hospital ward to provide cell pattern coverage, which consists of overlapping zones. Each repeater 218 has a transmission range of about 100 meters and is constructed to be wall mounted at any standard electrical outlet. Alternatively, each repeater 218 may be hardwired into the hospital's electrical grid system. The repeaters 218 are preferably spaced a sufficient distance apart from another so that each patient using the system will be within the range of multiple repeaters 218 at any given time.

In an exemplary embodiment, the repeater 218 may have a receiver, a signal conditioning unit, an error correction unit, a signal compressing unit, and a transmitter. The receiver receives the signals sent from the body electronics units 14. The signal conditioning unit may be used to amplify the signals, filter unwanted noise within the desired frequency range, or to remove common mode voltage errors. The signal compressor digitally compresses the digital signals to conserve bandwidth. The repeater 218 then bundles the digitally compressed signals received from multiple body electronics units 14 into discrete data packets and the transmitter transmits the data packets to a central monitoring station 220 via telemetry. Various air interfaces may be used to transmit the data packets from the repeater 218 to the central monitoring station 220, for example Bluetooth, IEEE 802.11b, WiFi, or other suitable wireless LAN system protocols.

The central monitoring station 220 may include multiple base stations 16 connected to monitors for displaying the physiological data an/or non-physiological data associated with each patient. The base stations 16 may have the same construction as previously described. For example, the base stations 16 may have, inter alia, a receiver, an A/C converter, and a demultiplexer. In addition, the base stations 16 may have a signal decompressor. The receiver receives the signals from the multiple repeaters 218. The signal decompressor decompresses the signals and the demultiplexer unbundles the data packets of information contained on each signal sent by each repeater 218. The A/D converter transforms the digital signal to an analog signal.

In one exemplary embodiment, central monitoring station 220 contains at least one base station 16 associated with each body electronics unit 14. The base station 16 may connect to a conventional monitor or display as described in detail above via snap terminals. Alternatively, the central monitoring station 220 may contain a central base station 222 that connects to a suitable monitor for displaying the physiological data an/or non-physiological data. The central base station 222 may be connected to a single monitor or multiple monitors via snap terminals (not shown) for displaying the physiological and/or non-physiological information pertaining to multiple patients. Alternatively, the central base station 222 may be hardwired to a single monitor or multiple monitors via a standard telemetry lead system. The central monitoring station 220 allows hospital personnel to remotely view and otherwise monitor the real time physiological data of the patients connected to the system. All of the waveform processing of the physiological data sent from the remote electronics unit 14 and relayed by the repeaters 218 is conducted in either the base station 16, the central base station 222, or the monitor.

In another embodiment of the present invention, the repeater 218 may transmit the data packets to a collection unit 224. The collection unit 224 gathers the multiple signals sent from multiple repeaters 218 and relays the signals to the central base station 222. The collection unit 224 may transmit the signals to the central base station via wireless LAN or wired link.

In the foregoing specification, the present invention has been described with reference to specific exemplary embodiments thereof. It will be apparent to those skilled in the art, that a person understanding this invention may conceive of changes or other embodiments or variations, which utilize the principles of this invention without departing from the broader spirit and scope of the invention. The specification and drawings are, therefore, to be regarded in an illustrative rather than restrictive sense. Accordingly, it is not intended that the invention be limited except as may be necessary in view of the appended claims.

We claim:

1. A system for monitoring the physiological data associated with at least one patient comprising, in combination:
at least one body electronics unit removably connected to a chest assembly having a plurality of sensors for acquiring physiological signals from a patient, the body electronics unit comprising a transmitter for transmitting the physiological signals;
at least one repeater comprising a receiver for receiving the physiological signals from the body electronics unit and a transmitter for transmitting the physiological signals;
at least one base station comprising a receiver for wirelessly receiving the physiological signals from the at least one repeater, the at least one base station capable of connecting to at least one monitor.

2. The system of claim 1 wherein the at least one repeater further comprises a signal conditioning unit, an error correction unit, and a signal compressing unit.

3. The system of claim 1 wherein the physiological signals transmitted between the at least one body electronics unit and the at least one repeater are transmitted utilizing the Bluetooth protocol.

4. The system of claim 1 wherein the physiological signals transmitted between the at least one repeater and between the at least one base station are transmitted utilizing the Bluetooth protocol.

5. The system of claim 1 wherein the at least one base station controls the data collected from the at least one body electronics unit.

6. The system of claim 1 wherein the at least one body electronics unit is capable of tagging each digital signal sent to the at least one repeater with an electronic identification number corresponding to the at least one body electronics unit.

7. The system of claim 1 further comprising a plurality of repeaters, the at least one body electronics unit further comprising a switch over protocol for establishing communications with the repeaters.

8. The system of claim 1 wherein the chest assembly further comprises:
a retaining section having a plurality of electrode connectors for removably connecting to the plurality of sensors;
a chest assembly connector attached to the retaining section; and
a sensor pin on the chest assembly connector for completing a circuit within the body electronics unit.

9. The system of claim 8 wherein the chest assembly further comprises:
a base layer having a first side and a second side, the first side attached to a plurality of electrically conductive elements, the second side attached to a shielding layer;
a first insulating layer positioned above the base layer;
a second insulating layer positioned below the base layer.

10. The system of claim 1 wherein the waveform processing of the physiological signals is conducted in the at least one base station.

11. The system of claim 1 wherein the waveform processing of the physiological signals is conducted in a monitor.

12. The system of claim 1 wherein the base station is capable of connecting to at least one monitor via snap terminals.

13. The system of claim 1 further comprising a plurality of body electronics units, the plurality of body electronics unit simultaneously transmitting physiological signals to the at least one repeater.

14. A system for collecting physiological data from at least one patient a plurality of patients comprising, in combination:
at least one body electronics unit removably connected to a chest assembly having a plurality of sensors for acquiring physiological signals from a patient, the body electronics unit comprising a transmitter for transmitting the physiological signals;
at least one repeater comprising a receiver for receiving the physiological signals from the body electronics unit and a transmitter for transmitting the physiological signals;
a central base station comprising a receiver for wirelessly receiving the physiological signals from the repeater, the central base station capable of connecting to at least one monitor.

15. The system of claim 14 wherein the at least one repeater further comprises a signal conditioning unit, an error correction unit, and a signal compressing unit.

16. The system of claim 14 wherein the physiological signals transmitted between the at least one body electronics unit and the at least one repeater are transmitted utilizing the Bluetooth protocol.

17. The system of claim 14 wherein the physiological signals transmitted between the at least one repeater and between the central base station are transmitted utilizing the Bluetooth protocol.

18. The system of claim 14 wherein the central base station controls the data collected from the at least one body electronics unit.

19. The system of claim 14 wherein the at least one body electronics unit is capable of tagging each digital signal sent to the at least one repeater with an electronic identification number corresponding to the at least one body electronics unit.

20. The system of claim 14 further comprising a plurality of repeaters, the at least one body electronics unit further comprising a switch over protocol for establishing communications with the repeaters.

21. The system of claim 14 wherein the waveform processing of the physiological signals is conducted in the at least one base station.

22. The system of claim 14 wherein the waveform processing of the physiological signals is conducted in a monitor.

23. The system of claim 14 wherein the chest assembly further comprises:
a retaining section having a plurality of electrode connectors for removably connecting to the plurality of sensors;
a chest assembly connector attached to the retaining section; and
a sensor pin on the chest assembly connector for completing a circuit within the body electronics unit.

24. The system of claim 23 wherein the chest assembly further comprises:
a base layer having a first side and a second side, the first side attached to a plurality of electrically conductive elements, the second side attached to a shielding layer;
a first insulating layer positioned above the base layer;
a second insulating layer positioned below the base layer.

25. The system of claim 14 wherein the central base station is capable of connecting to at least one monitor via snap terminals.

26. The system of claim 14 further comprising a plurality of body electronics units, the plurality of body electronics unit simultaneously transmitting physiological signals to the at least one repeater.

27. The system of claim 14 further comprising a plurality of repeaters, the plurality of repeaters simultaneously transmitting physiological signals to the central base station.
